(19) United States
(12) Patent Application Publication
(10) Pub. No.: US 2026/0232213 A1
(43) Pub. Date: Aug. 13, 2026

(54) PULSEWAVE VELOCITY DETECTION DEVICE

(71) Applicant: Quantum BioTek Inc., Atlanta, GA (US)

(72) Inventors: Astrid Androsch, Atlanta, GA (US); David Yuan, Atlanta, GA (US); Vladimir Fridman, Atlanta, GA (US)

(73) Assignee: Quantum BioTek Inc., Atlanta, GA (US)

(21) Appl. No.: 17/543,702

(22) Filed: Dec. 6, 2021

(51) Int. Cl. A61B 5/024; A61B 5/00; A61B 5/021; A61B 5/145; A61B 5/1455

(52) U.S. Cl. CPC A61B 5/02433; A61B 5/02108; A61B 5/02405; A61B 5/02427; A61B 5/14532; A61B 5/14552; A61B 5/6826; A61B 2562/0233

(57) ABSTRACT

A pulsewave velocity detection device provides a photodiode capable of detecting light transmission between 600-700 nm and 880-940 nm and a light emitting diode capable of transmitting the same. An optical isolation spacer separates the photodiode and the LED. A main control chip provides processing of collected pulsewave velocity signal detection by measuring with the photodetector. A microcontroller thereby calculates local metabolic rate, blood flow volume, Hb concentration, oxygen saturation, and corrected blood glucose and
correlates a corrected blood glucose value to the calculated local metabolic rate, blood flow volume, Hb concentration, and oxygen saturation. The instant abstract is neither intended to define the invention disclosed in this specification nor intended to limit the scope of the invention in any way.

### RELATED APPLICATIONS

The present invention is a Continuation in Part of U.S. Ser. No. 17/523,754, filed on Nov. 10, 2021 and incorporated by reference as if fully rewritten herein.

### COPYRIGHT NOTICE

Pursuant to 37 C.F.R. 1.71(d)-(e)(1988), a portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

### 1. Field of the Invention

The present invention relates generally to systems for the rapid screening and monitoring of cardiovascular disease and diabetes and, more particularly, to a novel pulsewave velocity detection device for use therein.

### 2. Description of the Related Art

The human body coordinates various physiological systems such as the cardiovascular neuromuscular, and excretory systems to communicate internally with each other and also with an external environment. However, there are cases where there is a malfunction within one of these systems that cause general, adverse health problems. Cardiovascular health problems caused by issues related directly or indirectly to the cardiovascular system have been one of the major, global, health concerns contributing majorly to the number of human deaths annually.

The basic function of arterial circulation is to supply body tissue with blood and to transfer pulse flow from the heart to stable flow in peripheral areas. Arterial blood pressure creates shaped waveforms when interacting between the stroke volume and the compliance of the large arteries and resistance of smaller arteries, also known as the “Windkessel effect”. Large arteries with a prevalence of elastic fibers in the arterial wall have the ability to adapt to the large pulse wave from the heart during the systolic period, in order to widen and store blood which may be supplied to tissues and organs during diastolic phase, creating compliance of the arterial system. The opposite is defined as arterial stiffness, which characterizes the degree of ageing of the arterial wall due to mechanical wear.

Pulse wave velocity increases with propagation to peripheral areas as it travels through vessels with an increased amount of collagen fibers in the arterial wall. This gradual increase of pulse wave towards the periphery is called pulse pressure amplification. Pulse wave ejected from the heart during the systolic phase is indicated as the primary pulse wave. In areas of increased resistance (structural or functional), many reverberations of the primary pulse wave occur. This results in a secondary pulse wave; this secondary wave moves through the arterial system in an opposite direction and interferes with the primary pulse wave. The resulting pulse wave and its shape in any segment of the arterial trunk are the result of summation of the primary and secondary waves.

The arterial stiffness index (ASI) is an overall measure of the health of a patient's cardiovascular system and has been identified as one predictor of cardiovascular disease morbidity and mortality. As the vessels become diseased or age with time, they become stiffer, increasing the pulse wave velocity and thus the arterial stiffness index. As such, the arterial stiffness index is a direct measure of the overall health of the cardiovascular system. Aortic stiffness is associated with atherosclerosis which may further impair ventricular perfusion, possibly leading to catastrophic reductions in ventricular function during ischemia. Aortic stiffening may increase the risk for the development of atherosclerosis. Arteriosclerosis is the hardening of the arteries, which can affect the heart, brain, kidneys, and extremities and constitute a main cause of morbidity and mortality.

Overall, cardiovascular disease accounts for 1 of every 3 deaths in the US and is the leading global cause of death. Cardiovascular disease also carries an astronomical toll to the healthcare system worldwide, and in the United States, including billions of dollars in healthcare costs annually, tremendous use of healthcare resources, and overall morbidity to the general population. However, there are no effective ways currently in the market that can accurately diagnose the early stages of cardiovascular disease. Current technology in cardiovascular disease detection has not been able to prevent coronary and peripheral artery disease, but rather is simply able to detect and monitor the damage comorbidities have on the cardiovascular system when they become severe and symptomatic.

Prediction of cardiovascular disease in early stages would thereby be greatly beneficial. As such it has the potential to be a great screening tool for coronary and peripheral artery disease. Pulse wave propagation can be described as an arterial wall disturbance caused by the ejection of the blood from the heart that propagates mainly toward the periphery. Pulsewave Velocity (“PWV”) and Arterial Stiffness Measurements are methods to measure regional arterial stiffness of the arterial territory between two measurement sites. This parameter is related not only to the elastic modulus of the arterial wall (which represents the intrinsic stiffness of the wall), but also to the arterial geometry (thickness and radius) and also blood density.

Some methods and devices are known that incorporate various mechanisms for measuring pulse wave propagation. For example:

U.S. Pat. No. 9,775,528, issued in the name of Vermeulen et al. discloses a photoplethysmography sensor apparatus and method that measures a photoplethysmographic signal without an ambient light interference. Ambient light signals are rejected by subtraction of a compensation current at the input of a transimpedance amplifier. The compensation current is controlled via a closed loop, without interfering with the low duty cycle operation of a photoplethysmography LED.

U.S. Patent Application Publication No. 2008/0177189, issued in the name of Kim et al., discloses a portable photoplethysmography measurement device. The device uses an improved photoplethysmography (PPG) sensor includes: a conductive member contacting with the skin of a user; a luminous element disposed on the conductive member to emit light towards the skin of the user through the conductive member; and a photo detector detecting the light emitted towards the skin of the user. An indium-tin oxide glass provides a skin contacting surface for the sensor.

U.S. Patent Application No. 2014/0005557, published in the name of Rich et al, discloses photoplethysmography (PPG) sensors, systems and accessories, and methods of making and using the same. The PPG sensors include a sensor body, a flex circuit attached or adjacent to the sensor body, and an elastomeric sleeve that envelops part of the sensor body and the flex circuit attached or adjacent thereto. The PPG sensor is particularly adapted for PPG monitoring at the nasal alar site.

U.S. Patent Application Publication No. 2021/0030290, published in the name of Lee et al, discloses an Electronic Device for Acquiring Biometrics Using PPG Sensor, and Method Therefor. A PPG sensor includes a light emitter that applies a current in a specified range and emits a light signal corresponding to the current and a light detector that amplifies a received light signal by applying one of a plurality of gain values. To obtain bio information from the PPG signal, the PPG signal needs to have an appropriate level. For example, when the PPG signal is saturated or a signal to noise ratio (SNR) is low, it may be difficult to extract bio information from the PPG signal. The level of the PPG signal may be proportional to the intensity of light emitted by the light emitter and the intensity of light collected by the light detector. The intensity of light emitted by the light emitter may be proportional to the intensity of a current applied to the light emitter. The light detector may amplify the collected light according to a predetermined gain value. The level of the acquired PPG signal may vary depending on the user even through a PPG sensor in which the current of the same intensity is applied and the same gain value is set. Therefore, a current intensity and a gain value for acquiring bio information from the PPG signal may vary for different users.

U.S. Patent Application Publication No. 2019/0328333, published in the name of Wijshoff et al., discloses a sensor device and method for sensing physiological information of a subject. The device and method include demodulating the modulated PPG signals, performing artifact-reduction on the demodulated PPG signals using the motion reference signal to obtain artifact-reduced PPG signals and modulating the artifact-reduced PPG signals on the carrier signals to obtain the output signal that is normalized by a motion reference signal representing motion of the body part. For photoplethysmography (PPG) signals are highly susceptible to motion which hampers their use in, e.g., activities of daily living (ADL), cardiopulmonary exercise testing (CPX), or cardiopulmonary resuscitation (CPR), the motion-reduction technique allows for more accurate sensing of physiological information.

U.S. Pat. No. 10,165,951, issued in the name of Sola i Caros et al., discloses a sensor device and method for measuring and determining a pulse arrival time (PAT) value. Determining a PAT value of a user using a sensor device comprising a photoplethysmographic (PPG) multichannel sensor formed from a plurality of PPG sensor channels and being adapted to measure a set of PPG signals, each PPG signal being measured by one of the PPG sensor channels when the multichannel PPG sensor is in contact with the user; comprising: measuring said set of PPG signals; extracting a plurality of features from each of the measured PPG signals; selecting a subset from the set of PPG signals based on the extracted features; and processing the selected subset of PPG signals to determine the PAT value. The disclosed sensor and method can be embedded into a chest belt and do not need skilled supervision. They can represent a potential candidate for the implantation of PWV measurement campaigns in the ambulatory setting.

U.S. Patent Application Publication No. 2020/0305738, published in the name of Genicot et al, discloses a computer-implemented method for direct photoplethysmography or direct PPG which is obtained by a time interval plurality of PPG signals for respective sensors in a wearable device. By combining the plurality of PPG signals a multi-sensor PPG signal is obtained.

European Patent Publication No. EP 3.841.966, published in the name of LeBoeuf et al., discloses physiological monitoring devices and methods using optical sensors configured with a photoplethysmography (PPG) sensor a photoplethysmography (PPG) sensor and a heater element configured to heat the skin of the subject at a location near the PPG sensor, thereby increasing blood perfusion.

U.S. Pat. No. 10,420,490, issued in the name of Rich et al., describes systems and methods for physiological monitoring using multiple signal processing devices in order to determine both blood oxygen saturation and rate of respiration.

U.S. Patent Application Publication No. 2021/0052175, published in the name of Stephens et al., discloses systems and methods for using characteristics of photoplethysmography (PPG) data to detect cardiac conditions with a wearable device. The signal waveform is modeled to be used to detect likely instances of cardiovascular conditions that affect blood flow during a cardiac cycle, including but not limited to atrial fibrillation, continuously with a wearable device.

U.S. Patent Application Publication No. 2019/0336081, published in the name of LeBoeuf et al., discloses physiological monitoring devices and methods using optical sensors having a photoplethysmography (PPG) sensor configured to detect/measure physiological information, namely subject stress.

U.S. Patent Application Publication No. 2016/0007862, published in the name of Ku, discloses a method for collecting personal health data and personal health device utilizing multiple photoplethysmogram (PPG) sensors configured for sensing multiple PPG signals. The electrodes are configured for sensing multiple skin voltages. The processing circuit is coupled to the PPG sensors and the electrodes and configured for estimating an ankle brachial pressure index (ABI) according to the PPG signals and the skin voltages.

U.S. Patent Application Publication Non. 2019/0350532, published in the name of LeBoeuf et al., discloses monitoring device including a photoplethysmography (PPG) sensor configured to determine heart rate and RR-interval (RRi) for the subject.

International Patent Publication WO 2012/074193, published in the name of Park et al., discloses a method for providing information for diagnosing arterial stiffness comprising the following steps: inputting user information, extracting feature points, and evaluating arterial stiffness. The extracting feature points comprises feature point correction, and evaluating arterial stiffness comprises the result of multiple linear regression analysis using brachial-ankle pulse wave velocity (baPWV) values.

And, International Patent Publication WO 2020/0176214, published in the name of Lee et al, discloses a finger cuff device with non-volume clamp, non-plethysmography pressure measurement method for continuous non-invasive blood pressure measurement. A photoplethysmograph simultaneously measures and records pulsatile blood flow through all the fingers of a patient's hand for the purpose of studying Raynaud's Phenomenon, also known as “vibration white fingers syndrome (VWF)”.

In spite of these many methods and devices for identifying PWV or measure arterial stiffness, none of these current technologies have resulted in an affordable and accessible diagnostic solution for detecting and monitoring the damage comorbidities have on the cardiovascular system in their early stages. What are currently available are expensive diagnostic tests that are poorly executed for daily practice. Carotid ultrasonography is an ultrasound-based diagnostic imaging technique to evaluate structural details of the two major blood vessels in the neck—the “carotid arteries”. Pulsewave Technologies in Austin, Texas provides a very large and expensive device that uses ankle brachial index and pulsewave velocity methodology. Non-contrast enhanced computed tomography (CT), also called calcium score heart scan, is currently used to find calcium deposits in arterial plaque of people with heart disease, but has no role in screening and is mostly used to diagnose when a blockage is already 80-90%. Additionally, such testing imparts high dosages of radiation to the patient and personnel. Coronary angiography is an invasive procedure that inserts a small tube into an arm vessel and threaded through to the aorta and into heart in order to perform tests.

Consequently, there are many unmet needs for practicing physicians to overcome high cost, equipment scarcity, the need for skilled personal or specialist, or lack of portability, improvements are needed for affordable detection and monitoring of cardiovascular diseases and the state of diabetes management in order to provide early intervention for disease regression.

### SUMMARY OF THE INVENTION

It is thus a general object of the present invention to provide a device for the rapid screening and monitoring of cardiovascular disease and diabetes.

It is a feature of the present to provide a novel pulsewave velocity detection device having a robust and optimal form factor for use therein.

Briefly described according to the present invention a new pulse detection module is provided that comprises a circuit board with an integrated pulse detection component. The pulse detection component that comprises a main control chip and a pulse acquisition unit for acquiring human pulse information. Having a smaller form factor and being more precise in pulse detection, the module is user-friendly and includes start and shutdown operations that are very responsive and fast. The pulse wave data detected may be used, inter alia, in the methods for non-invasively measuring glycated hemoglobin (HbA1C), arterial age or calcium score of cardiovascular, cholesterol, and diabetes-related parameters as disclosed in the Related Applications. Based on the different cycle methods used in combination with modeled artificial intelligence networks, various PPG features are extracted and further developed or analyzed. Based on preset cardiovascular parameters function, corresponding cardiovascular parameters of each cardiac cycle are obtained. The device provided, along with its operation software, allow for monitoring the status of a blood vessel by using several differential functions of the pulse wave and classifying types of the blood vessel flow. The optical finger-PPG sensing device with a biomedical sensing function measures various bio-information, pulse waves of blood vessels, and various indices to estimate the risks of arteriosclerosis. The light intensity signal of a finger as measured is used with time dynamics and analytics to evaluate the PPG signal changes for other related, biomedical applications.

According to another aspect of the present invention, the development of such non-invasive sensor may be further adapted for the measurement of glucose level directly through the measurement of oxygen saturation deduced through metabolic heat.

Advantages of the present invention allows for the acquisition of pulse waveforms, heart rate, and blood pressures, blood oxygen saturation, and vascular microcirculation parameters that can be directly outputted for direct monitoring or further analysis.

It is another advantage of the present invention to provide an integrated infrared dual-LED for use in blood oxygen measurement.

It is yet another advantage of the present invention to provide a light sensor with high sensitivity over a wide spectrum.

Further, the present invention provides an ultra-small form factor (i.e., 11.8 mm×5 mm), has an ultra-low power consumption, and utilizes a flexible and easy-to-use UART interface output.

Further objects, features, elements and advantages of the invention will become apparent in the course of the following description.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 depicts a QuanCardio™ finger-tip PPG (optical) device structure according to the preferred embodiment of the present invention;

FIGS. 2A, 2B and 2C are schematic representations of a top view, bottom view and side view thereof, respectively;

FIG. 3 is a schematic of the shell of the device of FIG. 1 and FIG. 2A-2C;

FIG. 4 is system configuration node diagram according to the preferred embodiment of the present invention;

FIG. 5 is a schematic block diagram depicting the operation of the QuanCardio™ finger-tip PPG (optical) device structure according to the preferred embodiment of the present invention;

FIG. 6 is a communication flowchart for a typical and exemplary operation of the preferred embodiment of the present invention; and

FIG. 7 is a device-software outline for a typical and exemplary operation of the preferred embodiment of the present invention.

### DESCRIPTION OF THE PREFERRED EMBODIMENTS

The best mode for carrying out the invention is presented in terms of its preferred embodiment, herein depicted within the Figures and Tables.

### 1. Detailed Description of the Figures

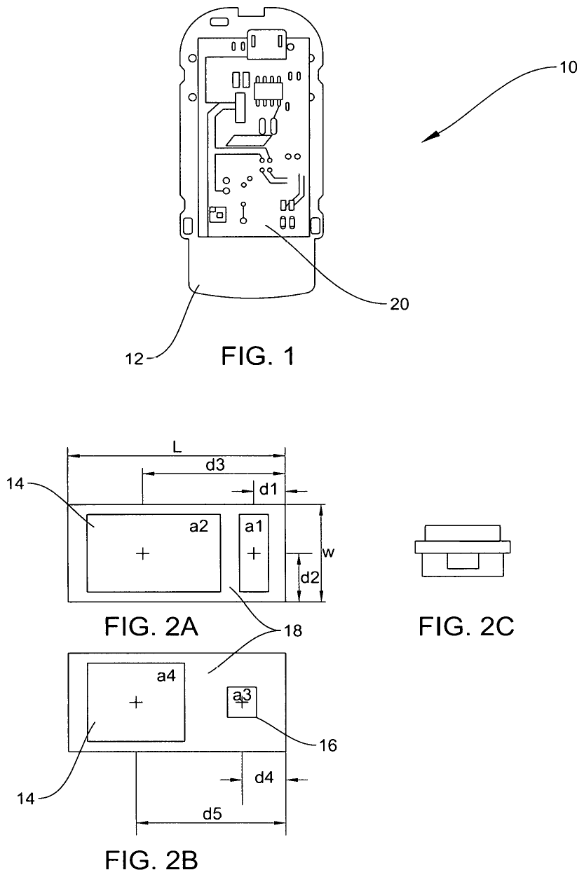

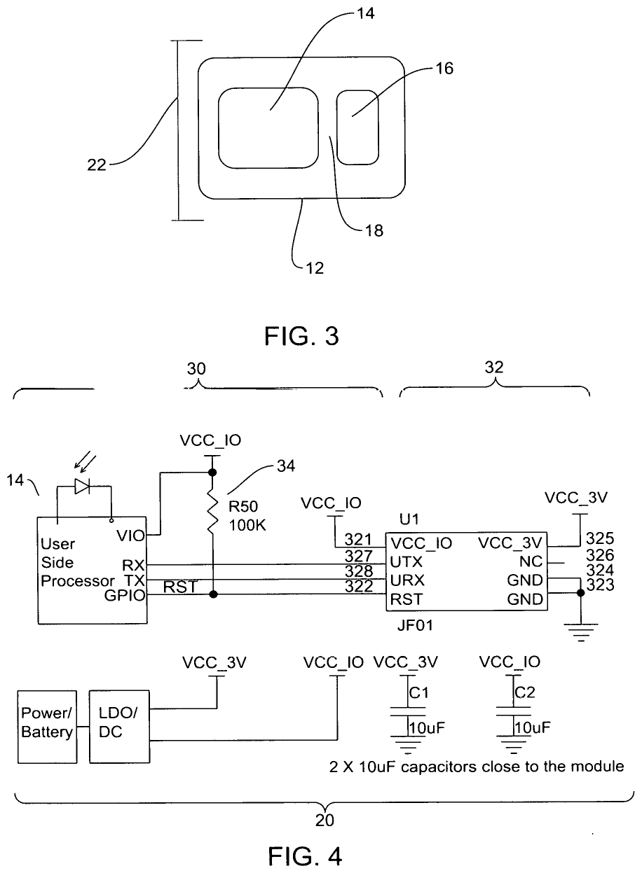

The present invention provides a system for use in various methods of disease screening which identifies volumetric blood flow pulsation signals. The preferred embodiment of the present invention, generally referred to herein as the “QuanCardio™” finger-type PPG (optical) sensor device, generally denoted as 10, is shown in FIG. 1 through FIG. 3. The sensor device 10 forms a pulse detection module which is highly integrated in size and operability. The sensor device 10 has a product housing 12 having a compact and integrated form factor. As shown best in conjunction with FIG. 2A through FIG. 2C, the housing 12 is formed of dimensions approximated by Table 1 below.

TABLE 1

| Reference of FIG. 2 | Dimensions in mm |
|---|---|
| Module length L | 11.84 ± 0.1 |
| Module width W | 5.0 ± 0.1 |
| Connector to edge d1 | 1.85 |
| Connector to edge d2 | 2.50 |
| Front device area a2 | 7.5*4.6 |
| Front device thickness Ta2 | 0.85 ± 0.05 |
| Connector thickness Ta1 | 0.8 ± 0.2 |
| Chip center to edge d3 | 7.87 |
| LED size a3 | 1.6*1.6 |
| LED to board edge d4 | 3.4 |
| LED thickness Ta3 | 0.6 |
| Sensor size a4 | (5.0 ± 0.15)*(4.24 ± 0.15) |
| Sensor to plate edge d5 | 8.1 |
| Sensor thickness Ta4 | 1.12 |
| PCB thickness Tpcb | 0.6 ± 0.1 |

The housing 12 comprises a photo diode sensor (“sensor”) 14 that is operatively controlling a light emitting diode (“LED”) 16, with a spacer 18 separating the LED 16 from the sensor 14. The spacer 18 may be black or opaque, and preferably would provide for non-transmission between the sensor 14 and the LED 16 in order to improve signal quality. The sensor 14 may comprise an integrated circuit 20. Within the housing 12 the integrated circuit 20 and LED 16 may be encased with a glass sheet 22. The glass sheet 22 protect the sensor and the sensor projection area needs to be well-connected to red and infrared light transmission materials. As such, the housing 12 and sensor 14 should preferably fit closely and, if necessary, filled with resin.

As best shown in conjunction with FIG. 4, a schematic of the integrated circuit 20 shows a main control chip 30 in operational connection with pulse acquisition unit 32, with the additional components described in conjunction with Table 2, with the electrical performance thereof best described in conjunction with Table 3.

TABLE 2

| Reference/ | | | |
|---|---|---|---|
| Part number | Signal Name | Function | Specified Examples |
| 321 | VCC_IO | Digital IO power | 2.6 V~3.6 V |
| 322 | RESETn | The module is reset | Low level effective |
| 323 | GND | Power supply | |
| 324 | GND | Power supply | |
| 325 | VCC_3V | LED power supply | 2.9 V~5.5 V |
| 326 | NC | retain | Must be suspended |
| 327 | UTX | Module UART sends | The level is consistent |
| | | | with the VDD IO |
| 328 | URX | Module UART | The level is consistent |
| | | receives | with the VDD IO |

TABLE 3

| Signal name | Voltage Range |
|---|---|
| VCC_3V | −0.3 V~6 V |
| VCC_10 | −0.3 V~3.8 V |
| URX | −0.3 V~(VCC_IO + 0. 3) V |
| RESETn | −0.3 V~(VCC_IO + 0. 3)V |

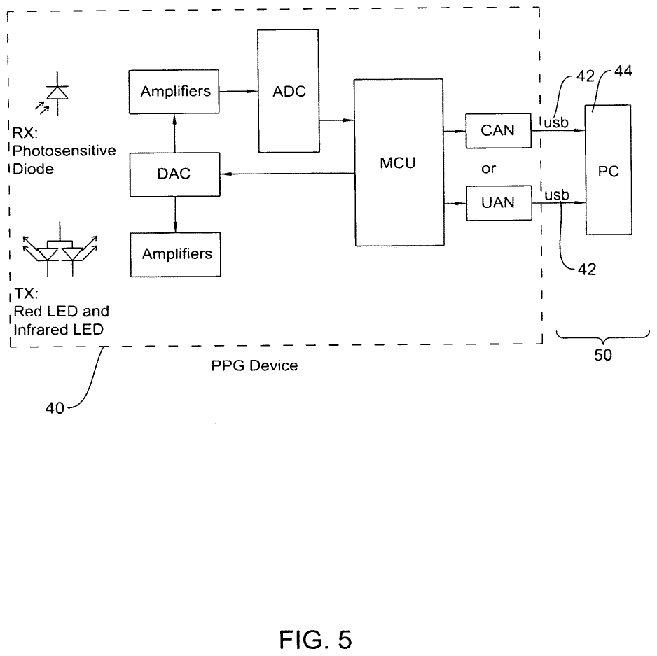

Referring in conjunction with FIG. 5, a schematic block diagram is shown depicting the operation of the QuanCardio™ finger-tip PPG (optical) device structure according to the preferred embodiment of the present invention. The device 10 includes an analog side 40 in communication with a digital side 50. The analog side 40 incorporates the optical module to act as scanner that detects the data and generates the pulsewave at a certain baud rate. The digital side 50 controls the power and manages the device 10. Analog data generated is communication 42 to a computer 44 where data may be upload and sent to the algorithm software on the computer 44 that does the analysis. The computer 44 may also contains USB and driver software.

The pulse acquisition 32 unit further comprises a photosensitive assembly, the photosensitive assembly comprises a photodiode 14 and a resistor 34. The resistor 34 is connected with the photodiode 14 in parallel, and two ends of the photodiode 14 are connected with the main control chip 20. The working principle involves the acquisition and processing of collected signals, calculating physiological indexes such as heart rate, blood oxygen saturation, microcirculation, and blood pressures, and the transmission of data waveforms through the universal asynchronous receiver transmitter (“UART”) interface. The QuanCardio™ device 10 of the present invention is thereby adapted for use as a piece of medical testing equipment, used mainly in cardiovascular chronic disease management and the monitoring of health abnormalities.

### 2. Operation of the Present Invention

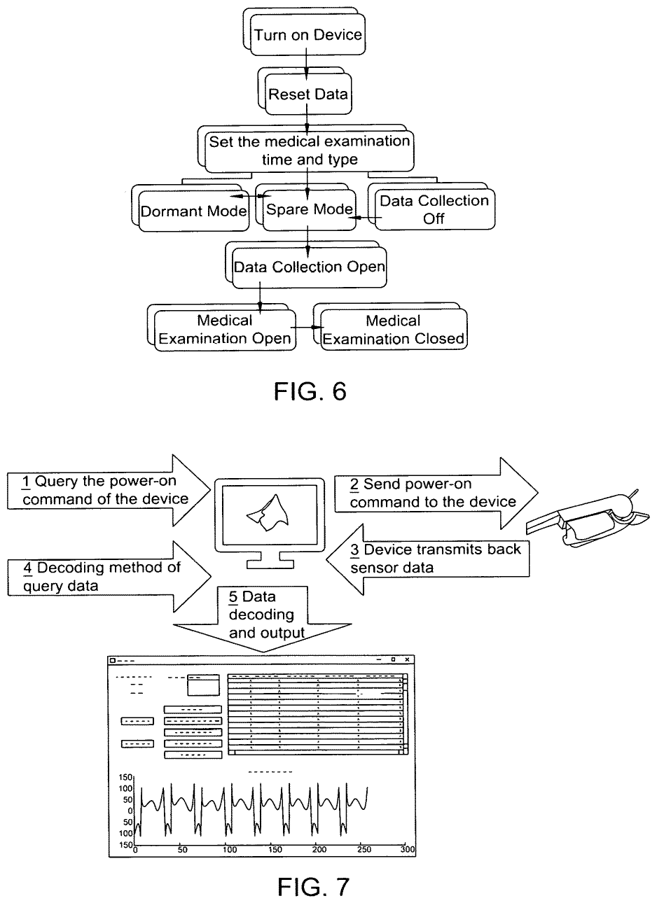

In the method of predicting the type of blood vessel from the acceleration pulse wave (as described in the Related Applications), the vascular health condition is calculated by the ratio of peak to the second derivative of blood volume pulse wave. However, to accurately diagnose the condition of blood vessels by the ratio of peak to the second derivative. Difficult cases may arise, and the results may vary with each measurement, resulting in poor reliability as a diagnostic device. Thus, there is a need for a method for more accurately and reliably diagnosing vascular conditions using acceleration pulse waves. In addition, there is a need to detect various health indices such as vascular age, vascular health index, and arteriosclerosis by detecting the state of blood vessels. Accordingly, as best shown in conjunction with FIG. 6 and FIG. 7. the present invention provides a method for detecting an accurate vascular health condition by detecting a waveform of a PPG signal and converting the waveform into a derivative function of various orders to quantify the degree of blood circulation.

In operation the improved pulse wave velocity detection device of the present invention may be uses as part of a wide variety of noninvasive diagnostic testing in a number of areas. These may include the following:
Cardiovascular health. Cardiovascular disease has become one of the principal diseases with high-risk related factors and effects such as smoking, hyperlipidemia, diabetes, damage of vascular wall structure and function, development of atherosclerosis, and arterial stiffness. The diagnosing and treating as early as possible of diseases are of great significance to all and involves the measurement of various indices and parameters related to the cardiovascular and circulatory system, the incidence of arteriosclerosis, chronic inflammation, blood clots, and stiff arteries (including veins and capillaries). QuanCardio™ aids clinicians in assessing cardiac, vascular, circulatory renal, and cerebral damages as well understanding the state of the disease to recommend treatment pathways and medications for disease management. Also, as a consumer health device, patients can self-measure details about their cardiovascular system, with minimal influence of medical personnel.
Diabetes. This focuses on glucose, as well as other related indices such as the HbA1c for the management and prediction of diabetes, and peripheral artery diseases.
Cholesterol. The device non-invasively measures various lipids concentrations and levels such as cholesterol.
Mental Health and Cardiac Stress. The applications of mental health are due to the measurement of various tolerance levels, cognitive abilities, and the effects on the cardiovascular system in the form of stress.
Covid-19 (Corona Virus). The device non-invasively measures the impact of Covid-19 on arterial function and structure; assessing increased arterial stiffness and lasting damage on the heart and circulatory system. It aids to assess cardiovascular complications such as obstruction of blood flow in the arterial and circulatory system. This includes acute cardiac injury, cardiac arrhythmias and endothelial damage leading to microvascular thrombosis and thromboembolic events, arrhythmia, acute coronary syndrome, and venous thromboembolism. The device aids clinicians to identify underlying health issues and the state of the blood vessels and comorbid cardiovascular diseases.
Vascular erectile dysfunction (impotence). Aiding clinicians to assess state of the disease and comprehensive care of patients with vascular erectile dysfunction to recommend treatment pathways and medications for disease management.
Skin Health Blood Flow Imaging. QuanCardio™ allows the measurement of blood flow within the skin and the related rhythms using a near-infrared PPG imaging system with the imaging techniques to deduce other applications for skincare, hair and scalp. The device also obtains insights into biological tissue perfusion and to study changes associated with wound healing and the formation of ulcers, and the imaging of the distribution of arterial oxygen saturation (SpO2) within a tissue. QuanCardio™ aids in recommending ingredients for skincare and haircare products as well as formulating cosmetics products.
Telemedicine and remote recording. The device uses metabolic heat as well as other parameters to remotely sense or measure biometric, health data.
Wearables such as gloves. QuanCardio™ technology into wearables can be applied in many areas, especially a built-in robotic glove or hand for the patient and elderly care.
Software embedded in robot for artificial doctor. QuanCardio™ sensors and software embedded into medical systems supported by tactile sensing can be applied in surgical robots and AI for surgery and other forms of medical services.
Military simulations, combat, emergency personnel, pilots. QuanCardio™ measures the vitals and cardiovascular health of personnel involved in combat or professional training, relevant and proper training in simulations, in real-life instances such as extreme exposure to pain, heat, or cold, high altitude, pressure, and extreme physical and mental challenges. The device also performs biometrics measurement for assessment and monitoring of health, stress, physical and cognitive performance, and tolerance levels. This has application for combat and security training (police, army, navy and military), emergency personnel, and air force and pilots.
Athletes. QuanCardio™ measures the vitals and vascular health of athletes involved in intensive training to evaluate physical and mental capacities, tolerance levels and aid in the preparation of individualized training plans and treatment pathways.
Post-surgery, pre-surgery, and during surgery. QuanCardio™ measures the vitals of patients with cardiovascular issues before, during, and after surgery to detect blood flow changes, monitoring of vitals and other related effects after treatment.
Skin and Hair health supplements. The various changes in skin blood flow, aid in recommended dosages of vitamins and supplements for health of skin and hair as well as hair growth and formulations for personalized vitamins and supplements.
Automotive. Capable of being built into a steering wheel of an autonomous driving vehicle, the QuanCardio™ has a built-in platform for drivers and pilots to measure real-time data of biometric vitals, with other beneficial applications such as car-to-health biometrics or sensor technology, health and mobility cockpit for in-cabin sensing to monitor health and measure vital signs in the connected vehicle. This is done through the different sensors that monitor the physical and mental states of the driver. QuanCardio™ also monitors with a built-in display on the dashboard to help drivers with heart problems and diabetes, people with heart conditions using their daily commutes as convenient checkups, tracking driver posture, breathing habits, heart rates, eye movements, facial feature recognition, alertness and emotional state for appropriate response from the car.
Pacemaker calibration. The use of cardiovascular parameters to calibrate pacemakers with the exact blood flow requirements for different patients.
Health supplements for general health, heart and vascular health. The measurement of various indices and parameters related to the circulatory and vascular system as well as general health assessment, aid in recommended dosages of supplements for the general, heart and vascular health and for the preparation of personalized vitamins and supplements.
Personalized, precision pharmaceuticals. The measurement of various indices and parameters related to the circulatory and vascular system as well as general health assessment, aid in tailoring pharmaceuticals and medical treatment to the individual characteristics of a patient and the preparation of precision pharmaceuticals for enhanced treatment outcomes.

The foregoing descriptions of specific embodiments of the present invention are presented for purposes of illustration and description. The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way. Any unintended embracement of such subject matter is hereby disclaimed. They are not intended to be exhaustive nor to limit the invention to precise forms disclosed and, obviously, many modifications and variations are possible in light of the above teaching. The embodiments are chosen and described in order to best explain principles of the invention and its practical application, to thereby enable others skilled in the art to best utilize the invention and its various embodiments with various modifications as are suited to the particular use contemplated. It is intended that a scope of the invention be defined broadly by the Drawings and Specification appended hereto and to their equivalents. Therefore, the scope of the invention is in no way to be limited only by any adverse inference under the rulings of Warner-Jenkinson Company, v. Hilton Davis Chemical, 520 US 17 (1997) or Festo Corp. v. Shoketsu Kinzoku Kogyo Kabushiki Co., 535 U.S. 722 (2002), or other similar caselaw or subsequent precedent should not be made if any future claims are added or amended subsequent to this patent application.

What is claimed is:

1. A pulsewave velocity detection device comprising:
a pulse acquisition unit comprising:
a photosensitive assembly comprising:
a photodiode capable of detecting red light transmission between 600 nm and 700 nm and near-infrared light transmission between 880 nm and 940 nm;
a resistor in electrical communication with the photodiode in parallel;
a light emitting diode capable of transmitting the red light and the near-infrared light;
a spacer separating the LED from the photodiode, said spacer providing optical isolation between the photodiode and the LED;
a main circuit board having a main control chip in operational connection with the pulse acquisition unit;
said main control chip adapted for processing of collected pulsewave velocity signals and transmitting of data waveforms through a universal asynchronous receiver transmitter interface; and
a housing containing the pulse acquisition unit, the spacer and the main circuit board, at least a portion of said housing be encased with a material optically transmissive at least to the red light and the near-infrared light, said transmission material in close optical connection with at least the photodetector and the LED.

2. The pulsewave velocity detection device of claim 1, wherein said housing is configured as a finger mounted sensor device and has a form factor no larger than about 12 mm in length and about 5 mm in width.

3. The pulsewave velocity detection device of claim 1, wherein said data waveforms transmitted through the universal asynchronous receiver transmitter interface are adapted for the identification of at least one physiological parameter selected from a group consisting of: levels of oxygen saturation in the blood; cardiac output; heart rate; heart rate variability; blood pressures; pulse pressures; respiration; lung capacity; vascular assessment; arterial diseases; arterial compliance; arterial aging; venous assessment; endothelial functions; microvascular blood flow or microcirculation; vasospastic conditions; autonomic function monitoring; vasomotor function and thermoregulation; orthostasis; other cardiovascular variability assessments.

4. The pulsewave velocity detection device of claim 3, wherein said data waveforms are filtered for motion artifacts prior to transmission.

5. The pulsewave velocity detection device of claim 1, wherein said data waveforms transmitted through the universal asynchronous receiver transmitter interface are adapted for the identification of at least one cardiovascular flow parameter selected from a first group consisting of: heart rate, pulse signal; systolic blood pressure; diastolic blood pressure; blood oxygen saturation; mean arterial pressure; and microcirculation within small vessels;

6. The pulsewave velocity detection device of claim 1, wherein said data waveforms transmitted through the universal asynchronous receiver transmitter interface are adapted for the identification of at least one predicted physiological parameters are used to estimate cardiovascular parameters selected from a group consisting of: Basic Cardio-related Parameters; Pulse Wave Velocity; Pulse Wave Analysis; Pulse Transit Time; Pulse Pressure Index; Arterial Conditions, Vascular Health and Stiffness Index; Estimated Arterial Age; and HbA1C.

7. The pulsewave velocity detection device of claim 1, wherein said LED is controlled to create a plurality of time spaced pulse points comprise a collection of sixty four (64) separate pulse points every 1.28 seconds.

8. A method for the noninvasive measurement of blood glucose levels comprising:
irradiating a skin surface of a finger with infrared light at an operative wavelength and at a fixed time series using a pulsewave velocity detection device of claim 1;
measuring a luminous intensity through the finger using the photodetector;
correcting the measurement of luminous intensity for resistance of clothes;
calculating local metabolic rate, blood flow volume, Hb concentration, oxygen saturation, and corrected blood glucose; and
correlating a corrected blood glucose value to the calculated local metabolic rate, blood flow volume, Hb concentration, and oxygen saturation.

9. The pulsewave velocity detection device of claim 2, wherein said data waveforms transmitted through the universal asynchronous receiver transmitter interface are adapted for the identification of at least one physiological parameter selected from a group consisting of: levels of oxygen saturation in the blood; cardiac output; heart rate; heart rate variability; blood pressures; pulse pressures; respiration; lung capacity; vascular assessment; arterial diseases; arterial compliance; arterial aging; venous assessment; endothelial functions; microvascular blood flow or microcirculation; vasospastic conditions; autonomic function monitoring; vasomotor function and thermoregulation; orthostasis; other cardiovascular variability assessments.

10. The pulsewave velocity detection device of claim 9, wherein said data waveforms are filtered for motion artifacts prior to transmission.

11. The pulsewave velocity detection device of claim 9, wherein said data waveforms transmitted through the universal asynchronous receiver transmitter interface are adapted for the identification of at least one cardiovascular flow parameter selected from a first group consisting of: heart rate, pulse signal; systolic blood pressure; diastolic blood pressure; blood oxygen saturation; mean arterial pressure; and microcirculation within small vessels;

12. The pulsewave velocity detection device of claim 9, wherein said data waveforms transmitted through the universal asynchronous receiver transmitter interface are adapted for the identification of at least one predicted physiological parameters are used to estimate cardiovascular parameters selected from a group consisting of: Basic Cardio-related Parameters; Pulse Wave Velocity; Pulse Wave Analysis; Pulse Transit Time; Pulse Pressure Index; Arterial Conditions, Vascular Health and Stiffness Index; Estimated Arterial Age; and HbA1C.

13. The pulsewave velocity detection device of claim 9, wherein said LED is controlled to create a plurality of time spaced pulse points comprise a collection of sixty four (64) separate pulse points every 1.28 seconds.

14. A method for the noninvasive measurement of blood glucose levels comprising:
irradiating a skin surface of a finger with infrared light at an operative wavelength and at a fixed time series using a pulsewave velocity detection device of claim 2;
measuring a luminous intensity through the finger using the photodetector;
correcting the measurement of luminous intensity for resistance of clothes;
calculating local metabolic rate, blood flow volume, Hb concentration, oxygen saturation, and corrected blood glucose; and
correlating a corrected blood glucose value to the calculated local metabolic rate, blood flow volume, Hb concentration, and oxygen saturation.
